(19) United States
(12) Patent Application Publication
(10) Pub. No.: US 2023/0337909 A1
(43) Pub. Date: Oct. 26, 2023

(54) DEVICE FOR RETINAL NEUROMODULATION THERAPY AND EXTRAFOVEAL READING IN SUBJECTS AFFECTED BY VISUAL IMPAIRMENT

(71) Applicant: ERA OPHTHALMICA S.R.L., Vicenza (IT)

(72) Inventors: Tommaso OCCHIPINTI, Vicenza (IT); Marco Ulises MORALES ARROYO, Padova (IT)

(21) Appl. No.: 18/028,954

(22) PCT Filed: Jan. 12, 2021
(86) PCT No.: PCT/EP2021/050496

§ 371 Date: Mar. 28, 2023

(30) Foreign Application Priority Data
Sep. 29, 2020 (IT) 102020000022876

(51) Int. Cl. A61B 3/113; A61B 3/032

(52) U.S. Cl. CPC A61B 3/113; A61B 3/032

(57) ABSTRACT

A device for retinal neuromodulation therapy and extrafoveal (eccentric) reading in subjects affected by visual impairment with loss of central vision, comprising a first apparatus designed to assess the eccentric reading capabilities correlated to the retinal anatomy of the patient; the first apparatus comprises a still camera or video camera device adapted to visualize the retina while the patient performs preset visual tasks; the device further comprises a second apparatus which comprises a screen associated with a tracking device for tracking the eyes which is adapted to monitor the eye movements of the patient, the second apparatus being adapted to control the images transmitted by the screen in order to execute eccentric exercises for visual rehabilitation.

This application is a U.S. national stage entry under 35 U.S.C. § 371 of PCT International Patent Application No. PCT/EP2021/050496, filed Jan. 12, 2021, which in turn claims priority to Italian Patent Application No. 102020000022876, filed Sep. 29, 2020, the contents of which are incorporated herein by reference in their entirety.

The present invention relates to a device for retinal neuromodulation therapy and extrafoveal (eccentric) reading in subjects affected by visual impairment with loss of central vision, for example as a consequence of macular degeneration as an adult and patients affected by amblyopia and by nystagmus as a child.

The term neuromodulation means the physiological process whereby a neuron uses chemical substances to control different populations of neurons. When the photoreceptors in the retina absorb light, they impart chemical and electrical impulses which in turn stimulate the nervous impulses, sending visual messages to the brain through the optic nerve. Therefore retinal neuromodulation is possible if the visual impulses are projected on the retina in a controlled environment.

The term visual impairment means a condition of reduced vision that can no longer be corrected with traditional lenses, or surgical means, which can make it very difficult to carry out the activities of daily life. It can be caused by various factors (congenital or acquired) and it can afflict both children and adults.

The principal cause of visual impairment in adults is represented by Age-related Macular Degeneration (AMD) which afflicts the elderly.

Age-related Macular Degeneration causes a reduction of central vision and people who are affected by it generally complain of difficulty in reading, writing, sewing or even simply recognizing the features of a face.

Generally, especially when the disease is in an advanced stage, it is extremely difficult to deal with the disease from the therapeutic point of view.

In these cases it is however possible to improve the autonomy of the person and their quality of life, by making use of visual rehabilitation so as to render the patient able to take best advantage of the residual vision in relation to their needs.

For example, if the patient presents a central scotoma, i.e. the central part of the retina (the macula), to a greater or lesser extent, has ceased to function, the person will notice a region which will be reported as a dark area or an area in which fixed objects disappear.

In these cases the patient needs to be trained to avoid these “blind spots” by seeking to bring the object of interest to a healthy region of the retina which is found all around the area afflicted by the disease. Several exercise techniques exist for obtaining this result, which can be performed at specialist centers and which are referred to as “training for eccentric vision”.

The training techniques used with children for nystagmus and amblyopia therapies are similar in terms of the definition of retinal rehabilitation with neurosensorial stimulation of the photoreceptors in selected zones of the retina, where the scope of retinal neuromodulation may be localized in zones of the central or paracentral retina depending on the degree of visual perception in different zones of the retina.

The use of eccentric areas for carrying out normal daily tasks is not automatic and necessitates a reorganization of the bodily movement in that one has to deal with an altered perception of space and distances which initially can create problems but which can be improved with other types of exercises for improving coordination.

The aids described above are not easy and spontaneous to use and they require an adaptation that is often difficult and which takes a long time. In the same way, the teaching of visual or behavioral strategies also requires a huge commitment in order to obtain the hoped-for result, i.e. the restoration of autonomy. For this reason the visual rehabilitation is an activity that is carried out by a team of professionals, normally in specialist clinics or at hospitals, who work with each other to take care of the various aspects, and who together evaluate the steps forward made by the patient.

U.S. Pat. No. 9,082,067 discloses the use of a miniaturized video camera mounted on an eyeglasses frame in order to capture an observation area (OA) and then, using an onboard microprocessor, is programmed to remap an electromechanical (MEMS) scanning projector or a DLP (Digital Light Processor) projector, display such observation area on a healthy region of the retina (PRL) in patients who have lost central vision as a result of the illness.

U.S. Pat. No. 6,705,726 discloses a device for examining the retina of the eye, a computer for controlling the optical instrument, an input device to allow the patient to enter a reaction during the examination, and an IR light for illuminating the retina.

The optical instrument is adapted to execute various types of examination.

In particular, during perimetry and microperimetry examinations, the display produces screens for the fixation of the eye of the patient and light stimuli to stimulate the eye of the patient. The light stimuli are selectable in terms of position and intensity. The input device is used to detect the reaction of the patient as the stimuli are seen. By choosing various retinal positions, information can be obtained about the sensitivity of the retinal region considered, and as a consequence a complete map of sensitivity can be obtained.

U.S. Pat. No. 3,883,235 relates to a computer program intended to be used to control the position, the size, the color and the intensity of test stimuli which are presented in predetermined positions in the field of view to the patient using appropriate devices.

The program determines the threshold level of a stimulus that the subject can detect at a given point of his or her field of view, by presenting the subject with such stimuli at a calculated intensity in pseudo-random or unpredictable positions on a field with substantially constant background, color and intensity.

The ocular movement of the subject is monitored and the relative position of the points is modified dynamically in order to take account of a coordinated translation of the point of fixation of the subject caused by movement of the eyes. Movement of the head, excessive movement of the eye or blinking of the eyelid are detected in order to allow the momentary interruption of the test.

U.S. Pat. No. 9,563,283 relates to an electronic device that comprises: a display; one or more gaze detecting sensors in order to determine a portion of the is display to which a user's gaze is currently directed; a timer for measuring periods of time associated with the user's current gaze at the display; and one or more processors adapted to: receive data relating to periods of time measured by the timer and determine therefrom a characteristic rate at which the user shifts his or her gaze from one part of the display to another; determine a portion of the display toward which the user's gaze was directed for a period of time longer than a period of time which is expected on the basis of his or her characteristic rate; identify an object included in the determined portion of the display; retrieve information that relates to the object identified; and enable the displaying of information based on the retrieved information.

Although both instruments and apparatuses for diagnosis and control of visual impairment, and programs and apparatuses adapted to rehabilitation, are known and quite well developed, it is a rather complex matter to adapt such apparatuses and such processes so as to enable patients to effect an extremely efficacious rehabilitation at home.

The aim of the present invention is to provide a device for retinal neuromodulation therapy and extrafoveal reading in subjects affected by visual impairment, by nystagmus, by amblyopia etc., which is capable of improving the known art in one or more of the above mentioned aspects.

Within this aim, an object of the invention is to provide a device for retinal neuromodulation therapy and extrafoveal reading in subjects affected by visual impairment, by nystagmus, by amblyopia etc., that is extremely effective and can be used by the patient without the need to visit clinics or hospital facilities.

Another object of the invention is to provide a device for retinal neuromodulation therapy and extrafoveal reading in subjects affected by visual impairment, by nystagmus, by amblyopia etc. that is highly reliable, easy to implement and of low cost.

This aim and these and other objects which will become better apparent hereinafter are achieved by a device for retinal neuromodulation therapy and extrafoveal reading in subjects affected by visual impairment, by nystagmus, by amblyopia etc., according to claim 1, optionally provided with one or more of the characteristics of the dependent claims.

Further characteristics and advantages of the invention will become better apparent from the description of some preferred, but not exclusive, embodiments of the device for retinal neuromodulation therapy and extrafoveal reading in subjects affected by visual impairment, by nystagmus, by amblyopia etc. according to the invention, which are illustrated for the purposes of non-limiting example in the accompanying drawings wherein:

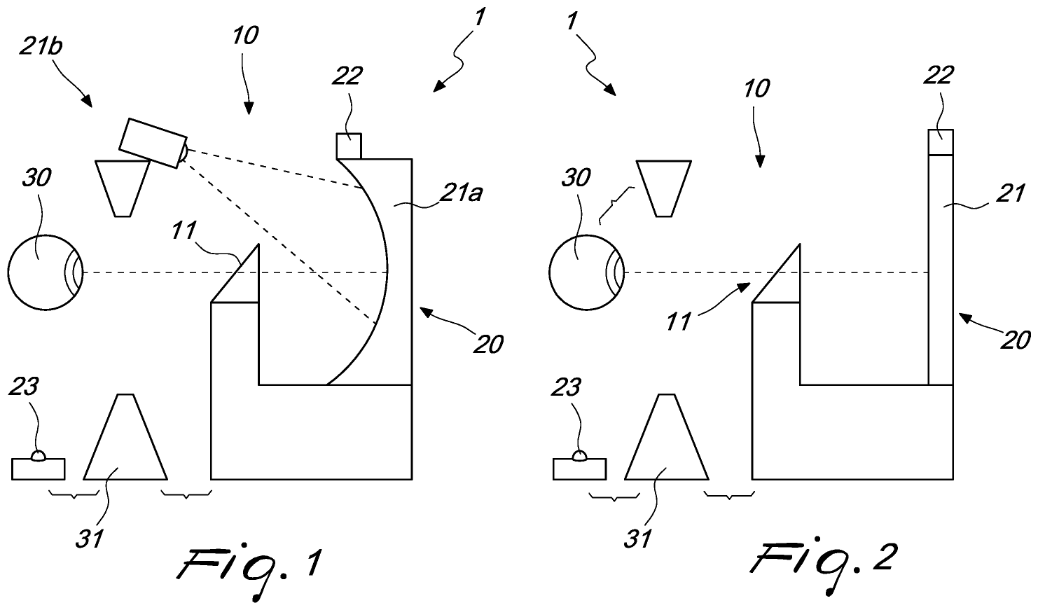

FIG. 1 is a schematic view of a first embodiment of the first apparatus of the device according to the invention;

FIG. 2 is a schematic view of a second embodiment of the first apparatus of the device according to the invention;

FIG. 3 is a schematic view of a first embodiment of the second apparatus of the device according to the invention;

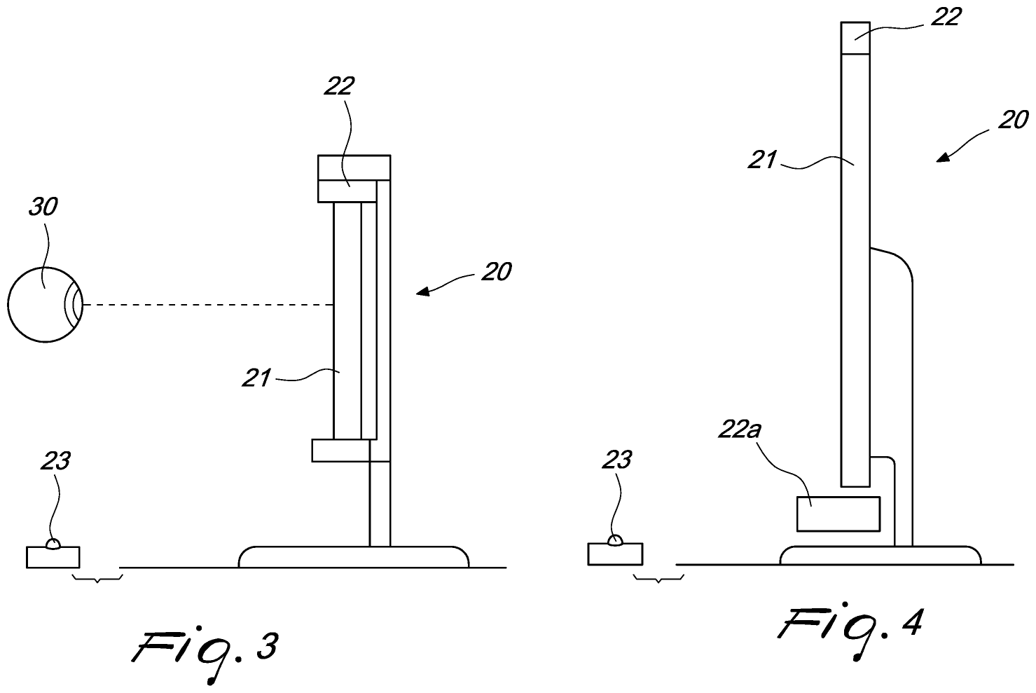

FIG. 4 is a schematic view of a second embodiment of the second apparatus of the device according to the invention.

With reference to FIGS. 1 and 2, the device for retinal neuromodulation therapy and extrafoveal (eccentric) reading in subjects affected by visual impairment with loss of central vision, according to the invention, generally designated by the reference numeral 1, comprises a first apparatus 10 designed to assess the central (foveal) and eccentric visual reading capabilities, correlated to the retinal anatomy of the patient, and a second apparatus 20 which comprises a screen 21 associated with a tracking device 22 for tracking the eyes 30.

In particular, the first apparatus 10 is typically used in clinics, consulting rooms or medical centers in the presence of a medical professional, while the second apparatus 20, of different types, as will be better explained below, can also be used at home, directly and autonomously is by the patient.

The first apparatus 10 comprises a still camera or video camera device 11, which is adapted to display the retina while the patient performs preset reading tasks.

In particular, the still camera or video camera device 11 is adapted to display the position of the pupil of the patient so as to align the system, follow the movements and display the retina while the patient performs the preset reading tasks.

The still camera or video camera device 11 has at least one sensor element for displaying the retina image and/or pupil size and position.

Specifically, the still camera or video camera device 11 comprises at least one sensor for displaying pupil size and position, and a processing device for identifying the position of the retina on the basis of the data collected by the sensor for displaying the position and for following the movements and pupil size.

Optionally the apparatus 10 can import images of the retina made by another type of instrumentation. Such images can be superimposed, rotated, aligned, scaled, related to and compared with the retinal images of the apparatus 10.

The first apparatus 10 is conveniently associated with a chin rest 31 which can be engaged by the head of the user.

The second apparatus 20 on the other hand has the tracking device 22, which is adapted to monitor the eye movements of the patient, and/or the movements and inclinations of the head of the patient.

In particular, the tracking device 22 is adapted to monitor the eye movements of the patient, and/or the movements and the inclinations of the head of the patient through measurements of gaze direction which can be obtained with, apart from other means, measurements of pupil movement and/or of first corneal reflection.

The tracking device 22, in addition to monitoring the eye movements is of the patient, and therefore measuring gaze direction, can be adapted to measure the distance between the head of the patient and the second apparatus 20, and/or to measure the inclination of the head of the patient with respect to the floor, and/or to measure other types of “vitiated” position of the head relative to the tracking device 22 and/or relative to the floor, through the use of at least one accelerometer and at least one gyroscope which are integrated in the device 22.

Preferably, the screen 21 and the tracking device 22, with particular reference to those used together with the first apparatus 10 in clinics, are adapted to operate at a working frequency at least equal to 100 Hz.

Even more advantageously, such screens 21 and the corresponding tracking device 22 are adapted to operate at a working frequency at least equal to 105 Hz.

With reference to what is shown in FIGS. 3 and 4, which represents a solution of the second apparatus which can be used at home, the screen 21 can comprise a monitor (for example of a television set as shown in FIG. 4, of a computer, of a tablet computer as shown in FIG. 3, of virtual reality or augmented reality devices etc.), or a projection surface 21a associated with a projector 21b (as shown in FIG. 1).

The second apparatus 20 is further adapted to control the images transmitted by the screen 21 in order to execute eccentric exercises for rehabilitation of eccentric reading and visual neuromodulation.

If the screen 21 is constituted by a tablet computer, this will incorporate the processor using the program and/or the application designed to manage the images.

If instead the screen 21 is constituted by a television or by a monitor, it will be possible to associate it with an apparatus for managing the images 22a which is functionally connected to the screen 21 of the television.

With reference to the second apparatus 20 for use at home, attention is again drawn to the fact that this is not associated with the first apparatus 10. Furthermore, the second apparatus 20 for use at home can also operate at a working frequency less than 105 Hz or at 100 Hz.

According to the present invention, the device 1 comprises a therapeutic device for the rehabilitation of subjects affected by visual impairment.

Preferably, the device 1 comprises a device for storing and controlling the data acquired by the first apparatus 10.

Advantageously, the first apparatus 10 comprises an infrared video camera which defines the still camera or video camera device 11.

Alternatively, the first apparatus 10 can be constituted by an apparatus for tomographic retina measurement (OCT).

The infrared video camera is adapted to monitor the simultaneous image of the retina of both eyes 30 of the patient.

The infrared video camera is further associated with a pupil tracker, monocular or preferably binocular, which is adapted to recognize the movement of the first corneal reflection and/or the pupil size, and with a detector of the position of the center of the pupil of the patient which is adapted to emit audio signals as a feedback (biofeedback) function in order to alert when the eye 30 of the patient moves away from an assigned position.

Such audio signals can comprise intermittent or constant tones, and they can also be constituted by music tracks constituted by sounds and music tracks in 3D audio format as well.

Conveniently, the second apparatus 20 comprises the screen 21 on which different images are projected with the goal of being viewed in binocular mode or monocular mode by the patient.

Such images can be in the form of letters, symbols, drawings, videos and in various colors, intensities and dimensions, and video games controlled manually or controlled with the eye movements of the patients.

Conveniently, the position of the images projected on the screen 21 is substantially at the anatomical visual axis or in different eccentric positions of the retina that correspond to different viewing angles.

The first apparatus 10 is intended to measure:
eccentric reading acuity (ERA);
eccentric reading speed (DRS);
eccentric visual acuity (EVA);
retinal scotometry (ResCo); and
dynamic fixation training (DyFix);
pupillary reading response (PuResp).

Advantageously, the second apparatus 20 comprises a screen, particularly chosen from the group comprising a TV, a PC monitor, an iPad, an Android tablet, and a tracking device 22 for each eye 30.

Furthermore, the patient can optionally wear lenses, ophthalmic or contact, to correct refraction (sphere and cylinder).

Conveniently, the second apparatus 20 is provided with a device for entering data relating to the eccentric reading and to the retinal neuromodulation to be performed.

Such second apparatus 20 is further adapted to recognize the position of the patient, the center of the pupil and/or the size of the pupil, and is adapted to monitor and record the movements of the eyes 30.

In this regard, the second apparatus 20 is advantageously associated with a device for detecting the distance of the face of the patient from the screen 21, and/or of the inclination and any vitiated position of the head.

Such detection device can likewise be connected functionally to a user interface device which is designed to indicate the correct distance to the patient at which to perform the exercises.

The second apparatus 20 is associated with a locator device 23, for example constituted by a button, which can be actuated by the patient in response to various visual stimuli (images, letters, words, videos).

Similarly a voice recognition device can be provided for evaluating is reading speed and efficacy: for the purposes of example, through one or more microphones, the performance levels of speed and efficacy of reading the text can be measured.

Furthermore devices can be provided which are adapted to measure reading speed based on the use of algorithms that recognize the direction and movement of gaze, in particular saccadic movement when the gazes settles on and advances over the letters of a word or phrase.

Furthermore, the second apparatus 20 comprises a software program designed to compare the response of the patient using the locator device 23 with standard values in order to evaluate eccentric reading capabilities.

Conveniently, the second apparatus 20 is provided with an analysis device for analyzing, during the visual exercise, the simultaneous movements of each eye 30 of the patient that are detected by the tracking device 22.

Use of the device 1 according to the invention is the following.

Partially-sighted patients carry out, by virtue of the first apparatus 1, typically at a clinic or a doctor's surgery, an evaluation of eccentric reading capabilities correlated to retinal anatomy and/or to pupil size.

In this manner, the doctor is able to display the state of the retina of the eyes 30 of the patient while the patient performs the preset visual tasks.

The doctor, or a dedicated program, determines, in light of the state of the retina, a series of exercises for the evaluation and the rehabilitation of eccentric reading, which can be carried out at home using a screen 21 of the second apparatus 20.

Such screen 21 is likewise associated with a tracking device 22 for tracking the eyes 30, which is adapted to monitor, at any frequency, but in particular with a frequency equal to at least 100 Hz, and more specifically equal to at least 105 Hz, the eye movements of the patient through, for example, pupil movement and first corneal reflection.

In practice it has been found that the invention fully achieves the is intended aim and objects by providing a device 1 for the evaluation and rehabilitation of eccentric reading in subjects affected by visual impairment which is extremely effective and which makes it possible to perform the visual rehabilitation exercises at home.

The invention thus conceived is susceptible of numerous modifications and variations, all of which are within the scope of the appended claims. Moreover, all the details may be substituted by other, technically equivalent elements.

In practice the materials employed, provided they are compatible with the specific use, and the contingent dimensions and shapes, may be any according to requirements and to the state of the art.

The disclosures in Italian Patent Application No. 102020000022876 from which this application claims priority are incorporated herein by reference.

1-15. (canceled)

16. A device for retinal neuromodulation therapy and extrafoveal (eccentric) reading in subjects affected by visual impairment with loss of central vision, comprising a first apparatus designed to assess eccentric reading capabilities correlated to a retinal anatomy of a patient, said first apparatus comprising a still camera or video camera device adapted to visualize the retina while said patient performs preset visual tasks, said device further comprising a second apparatus which comprises a screen associated with a tracking device for tracking the eyes which is adapted to monitor eye movements of said patient, said second apparatus being adapted to control images transmitted by said screen in order to execute eccentric exercises for visual rehabilitation.

17. The device according to claim 16, wherein the still camera or video camera device comprises at least one sensor for displaying pupil size and position.

18. The device according to claim 16, wherein said tracking device for tracking the eyes is adapted to monitor the eye movements of said patient by way of pupil movement and/or a first corneal reflection.

19. The device according to claim 16, wherein said screen and said tracking device are adapted to operate at a working frequency at least equal to 105 Hz.

20. The device according to claim 16, further comprising a device for storing and controlling data acquired by said first apparatus.

21. The device according to claim 18, wherein said first apparatus comprises an infrared video camera which defines said still camera or video camera device, said infrared video camera being adapted to monitor a simultaneous image of the retina of both eyes of said patient, a binocular pupil tracker adapted to recognize a movement of the first corneal reflection, and a detector of a position of a center of the pupil of said patient which is adapted to emit audio signals as a feedback function in order to alert when the eye of said patient moves away from an assigned position.

22. The device according to claim 16, wherein said first apparatus comprises an apparatus for tomographic retina measurement (OCT).

23. The device according to claim 16, wherein said second apparatus comprises said screen on which different images are projected with the goal of being viewed in binocular mode or monocular mode by said patient, said images being in the form of letters, symbols, drawings, videos and in various colors, intensities and dimensions.

24. The device according to claim 23, wherein a position of the different images projected on said screen is substantially at an anatomical visual axis or in different eccentric positions of the retina that correspond to different viewing angles.

25. The device according to claim 16, wherein said first apparatus is intended to measure:
eccentric reading acuity (ERA);
eccentric reading speed (DRS);
eccentric visual acuity (EVA);
retinal scotometry (ResCo);
dynamic fixation training (DyFix); and
pupillary reading response (PuResp).

26. The device according to claim 16, wherein said second apparatus comprises said screen, particularly chosen from the group comprising a TV, a PC monitor, an iPad, an Android tablet, a wearable lens, and a tracking device for each eye.

27. The device according to claim 16, wherein said second apparatus is provided with a device for entering data relating to the eccentric visual training to be performed, said second apparatus being adapted to recognize a position of the patient, a center of the pupil, and being adapted to monitor and record the movements of the eyes.

28. The device according to claim 16, wherein said second apparatus is associated with a locator device which can be actuated by the patient in response to various visual stimuli (images, letters, words, videos).

29. The device according to claim 28, wherein said second apparatus comprises a software program designed to compare a response of the patient using said locator device with standard values in order to evaluate eccentric reading capabilities.

30. The device according to claim 16, wherein said second apparatus is provided with an analysis device for analyzing, during a visual exercise, the movements of each eye of the patient that are detected by said tracking device.
